(19) United States
(12) Patent Application Publication
(10) Pub. No.: US 2024/0378764 A1
(43) Pub. Date: Nov. 14, 2024

(54) METHOD FOR CREATING SYNTHETIC DATA FOR AI MODEL TRAINING

(71) Applicant: KARL STORZ SE & Co. KG, Tuttlingen (DE)

(72) Inventors: Igor KHAZATSKIY, Tuttlingen (DE); Andrej ADERHOLD, Tuttlingen (DE); Alexander BALASHOV, Tuttlingen (DE)

(73) Assignee: KARL STORZ SE & Co. KG, Tuttlingen (DE)

(21) Appl. No.: 18/658,496

(22) Filed: May 8, 2024

(30) Foreign Application Priority Data
May 10, 2023 (DE) 10 2023 112 359.9

(51) Int. Cl. G06T 11/00; G06V 10/774; G16H 30/40

(52) U.S. Cl. CPC G06T 11/00; G16H 30/40; G06V 10/774

(57) ABSTRACT

A computer-implemented method for creating application-specific data for training artificial intelligence-based object recognition, comprising the creation of a command file for execution on a processor for the purpose of creating an image data record using at least one configuration file, the creation of at least one configuration file describing a scene to be simulated, the transfer of the command file and the configuration file to and the execution of these files on the processor for the purpose of creating the image data record and an annotation file associated with the image data record, and the storage of the image data record together with the annotation file.

### CROSS-REFERENCE TO RELATED APPLICATIONS

This application claims priority of German Patent Application No. DE 10 2023 112 359.9 filed on May 10, 2023, the contents of which are incorporated herein.

### TECHNICAL FIELD

The present disclosure relates to methods for creating synthetic training data for training artificial intelligence-based models.

### BACKGROUND

Machine learning is the field of artificial intelligence which considers the independent inference of relationships on the basis of exemplary data. In this context, algorithms are “fed” structured data; thus, these algorithms learn from the data and can make predictions on the basis thereof. These algorithms work on the principle of creating a mathematical model from input data, this model then allowing the algorithms to make data-controlled predictions or decisions.

Supervised learning algorithms in particular may offer promising solutions to many real problems, such as text classification, object classification and recognition, medical diagnoses, and information security. To make supervised learning work, there is a need for a data record from which the model can learn to make correct decisions.

A significant limitation of supervised learning in real applications lies in the difficulty of obtaining data for training the prediction models. The classification power of a prediction model is known to depend decisively on the quality of the training data. Ideally, classifiers are trained using diverse, structured data which represent all classes in full; in addition to the selection of the suitable data, this also requires a corresponding scope.

However, there are cases where the problem to be solved relates to a niche area with little data available, or cases where the availability of data is restricted for other, e.g., regulatory reasons, and the procurement of the correct data record per se represents a challenge. Examples in this respect include in particular methods for object recognition in the medical and/or clinical field, for example with regards to monitoring or assisting surgical procedures, where little to no data are available, not least on account of data protection requirements.

However, insufficient data available for a specific scenario may lead to the predictions of the model being inaccurate or distorted. There are options such as data augmentation and data labeling which may assist with rectifying the defects, but the result might still not be accurate or reliable enough.

Accordingly, variations can for example be created by systematic modifications on the basis of real data, for example image data, and the data record can be augmented in this way. Additionally, the prior art has also disclosed approaches for the creation of synthetic data. The lack of diversity of the data is often problematic in this case and may lead to inaccurate prediction models.

Data labeling refers to the process of identifying raw data (images, text files, videos, etc.) and adding one or more meaningful and informative labels in order to provide context so that a machine learning model can learn therefrom. For example, such labels could specify whether a photograph contains a bird or an automobile, the words uttered in an audio recording, or whether an x-ray image contains a tumor.

Data labeling usually starts with humans making appropriate judgments with regards to unstructured data in order thus to structure these. Labeling can be as coarse as a simple yes/no or as granular as the identification of the specific pixels in an image which are assigned to a specific object. Accordingly, the labeling of raw data is as complicated and costly as desired—depending on the granularity and quality required.

The machine learning model uses these labels in order to learn the underlying patterns. The result is a trained model which can be used to make predictions in relation to new data. The accuracy of its trained model depends on the accuracy of the labeled data record. The quality and hence applicability of the trained model is limited if insufficient data with appropriate labeling are available, and the statements provided by the model might not be reliable.

Accordingly, it is an object of the present disclosure to at least partly overcome the limitations or deficiencies of the methods known from the prior art.

### SUMMARY

It is the object of the present disclosure to at least partly overcome the disadvantages known from the prior art. The present object is achieved by the methods according to the disclosure as claimed in claims 1, 13, and 14. Preferred configurations of the disclosure are the subject matter of the corresponding dependent claims.

Accordingly, the present disclosure discloses a computer-implemented method for creating application-specific data for training artificial intelligence-based object recognition in images of medical and/or clinical workflows, comprising the creation of a command file for execution on a processor to create an image data record comprising up to three dimensions using at least one configuration file, the creation of at least one configuration file describing a scene to be simulated, the configuration describing at least one camera and at least one light source, the transfer of the command file and the configuration file to and the execution of these files on the processor for the purpose of creating the image data record, the image data record corresponding in terms of perspective to the at least one camera and taking into account the at least one light source, the creation of an annotation file associated with the image data record, and the storage of the image data record together with the annotation file.

In preferred embodiments of the method according to the disclosure, the configuration file can describe a static or dynamic scene.

In preferred embodiments of the method according to the disclosure, the configuration file can comprise general parameters, objects, materials, illumination, camera properties, object positions, object movements, properties of the surroundings, occlusion planes, and/or time-dependent parameter changes. Optionally, the configuration file may refer to objects and/or materials in a non-transitory memory, which are added for the purpose of creating the image data record when the command file is executed. These objects may be stored in an appropriate database and may be included in this form in the creation of the scene as retrievable descriptions and/or pieces of image information.

In preferred embodiments of the method according to the disclosure, the scene described in the configuration file, for example intending to represent a medical and/or clinical workflow, can be simulated in predetermined surroundings, the predetermined surroundings describing a real room. In this context, workflows can be specific medical procedures or else specific processes in a certain area of a clinic, for example a specific operation in an operating theater, or else the preparation or even cleaning of an operating theater. The predetermined surroundings may be stored in a database in the form of a description and/or image data. For example, a detailed description of an operating theater with accurate size information and optionally with appropriate photographs may be stored in a database. The user can select the appropriate surroundings, for example a specific operating theater, during the scene description so that the generated scene is then executed in these selected surroundings. In this context, the description of the surroundings or of the real room may be captured by way of a scanning device, for example a Light Detection and Ranging (“lidar”) scanner, and/or be constructed from digital images in embodiments of the disclosure. In particular, these pieces of information may also contain information about (room) cameras present. The scenes created by the method are then based on the surroundings information as fixed parameters, whereby the specificity of the data for the training of object recognition algorithms in the specific field can be increased significantly.

In preferred embodiments of the method according to the disclosure, the general parameters can comprise a description of the scene to be simulated. In particular, specific classes of scenes to be simulated can also be determined and selected by the user in this context. For example, staying with the example of an operating theater, certain types of operations (for example aligned with the region of the body to be operated on) can be determined with respective specific, recurring properties (for example related to the position of the surgeon).

In preferred embodiments of the method according to the disclosure, further image data records and annotation files associated with the further image data records can be created on the basis of variations of the descriptions contained in the configuration file. In this context, the configuration file in particular may contain definitions regarding the variations intended to be carried out or the possible variations. Thus, in embodiments of the method, the configuration file can contain allowed and forbidden states of the objects and illumination, and the variations can be defined by these states. Accordingly, the variations may contain changes in the light source(s) or in the illumination and/or in the formation of shadows. As an alternative to that or in addition, the variations may relate to the presence, position, and/or movement of various objects.

In preferred embodiments of the method according to the disclosure, the image data records created and the annotation files associated therewith can be stored in categorized fashion.

In preferred embodiments of the method according to the disclosure, the configuration file, at least in part, may contain pieces of information based on a default. In particular, defaults may contain general, recurring properties of a scene to be simulated. In an alternative to that or in addition, the defaults may also specify certain configurations and/or properties of objects and/or materials and thus for example restrict the possible variations and/or the selection of objects and/or materials from the database. For example, the color of the work apparel of the clinical staff may be selected in advance by way of a default that determines a specific hospital. This allows the generated image data to be specified further and—by way of the use as a training data for object recognition—the resultant object recognition to be improved.

In a further aspect, the disclosure relates to a method for creating an application-specific, artificial intelligence-based object recognition, comprising the training of a prediction algorithm with training data created according to the above-described method.

In a further aspect, the disclosure relates to a method for recognizing objects in a specific application, wherein a prediction algorithm trained with training data created according to one of the above-described methods is applied to at least one digital image and wherein the at least one image is an image of a scene or has any other property optionally determined by way of defaults in the configuration file, the at least one image forming the basis of the training data created.

In preferred embodiments of the method according to the disclosure for recognizing objects in a specific application, the specifically trained prediction algorithm is selected on the basis of the recognition of the specific application. For example, by reading a room camera, a specific room can be recognized and the corresponding algorithm, trained for this room, can be selected. In an alternative to that or in addition, other pieces of information, for example operating theater scheduling, can be used as a basis for acquiring the type of procedure and can be used to select the corresponding algorithm trained for this procedure. In this case, depending on the specification of the training data created according to the disclosure, all underlying parameters can be used for the selection of specific algorithms.

### BRIEF DESCRIPTION OF THE DRAWINGS

FIG. 1 shows a schematic illustration of the method.

FIGS. 2a and 2b show created image data.

The attached drawings elucidate exemplary embodiments of the disclosure and serve the exemplary explanation of the fundamentals of the disclosure.

### DETAILED DESCRIPTION OF PREFERRED EMBODIMENTS

The present disclosure will now be described in detail with reference to the attached drawings. However, the disclosure can be embodied in many different forms and should not be construed as restricted to the embodiments presented here. It should be observed that the drawings elucidate general features of the methods used in the respective embodiments. However, these drawings might not exactly reproduce the exact structure or the exact feature of a given embodiment. Moreover, identical reference signs in the drawings denote corresponding parts in all the various views or embodiments.

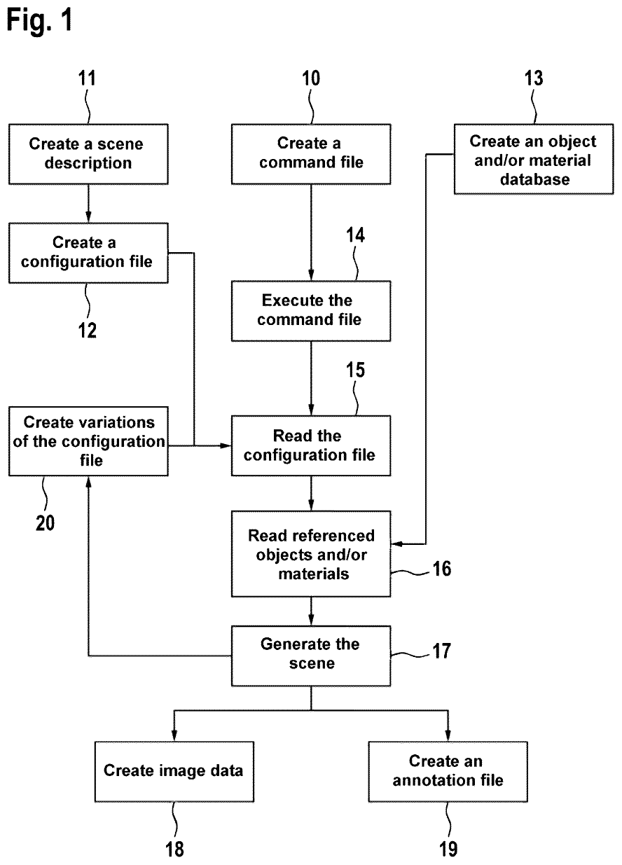

Here, FIG. 1 shows a schematic illustration of the method according to the disclosure. In a first step 10, a command file is created for execution on a processor to create an image data record comprising up to three dimensions using at least one configuration file. Depending on the application, the command file may be created in advance and may be used for various implementations of the method according to the disclosure. By preference, an appropriate processor-executable computer program, in particular a computer program for creating so-called “computer-generated imagery” (CGI), for example a blender, is preferably used for the creation of the image data record by the processor. Accordingly, the command syntax to be used in the command file depends on what command syntax the processor is able to process in conjunction with the computer program. For example, a command file may be based on, e.g., Python™, like in the following example code.

```
import os
import json
# Blender 3.4.1 Python API https://docs.blender.org/api/current/index.html
import bpy
from mathutils import Matrix
# Download model from Artifact repository
from artirepo import connect_repo
from blender_scene import create_annotation
# --- Basic definitions ---
repo_url = ‘https://url.to.blender.artifact.repo’
output_dir = ‘Rendered_Images/’
if not os.path.isdir(output_dir):
  os.mkdir(output_dir)
render_file_pattern=‘or_sample_%s_%d.jpg’
anno_file_pattern=‘or_sample_%s_%d_labels.xml’
local_asset_store = dict( )
# --- Setting up Blender render engine ---
bpy.context.scene.render.resolution_percentage = 100
bpy.data.scenes[0].render.engine = “CYCLES”
bpy.context.preferences.addons[“cycles”].preferences.compute_device_type = “CUDA”
bpy.context.scene.cycles.device = “GPU”
bpy.context.preferences.addons[“cycles”].preferences.get_devices( )
print(bpy.context.preferences.addons[“cycles”].preferences.compute_device_type)
for d in bpy.context.preferences.addons[“cycles”].preferences.devices:
  d[“use”] = 1 # Using all devices, include GPU and CPU
# --- Methods to create scene
def load_asset(uuid, url = None):
  conn = connect_repo(url)
  asset = conn.get_asset(uuid=uuid)
  if asset is None:
    return None
  return asset
def create_room(room):
  bpy.ops.mesh.primitive_cube_add(location=(0.0, 0.0, 0.0))
  cube = bpy.context.selected_objects[0]
  cube.name = “or”
  cube.dimensions[0] = room[‘dim’][‘x’][1] − room[‘dim’][‘x’][0]
  cube.dimensions[1] = room[‘dim’][‘y’][1] − room[‘dim’][‘y’][0]
  cube.dimensions[2] = room[‘dim’][‘z’][1] − room[‘dim’][‘z’][0]
  # translate cube to match initial dim
  translation = (
    room[‘dim’][‘x’][1] − abs(room[‘dim’][‘x’][1] − room[‘dim’][‘x’][0]) / 2,
    room[‘dim’][‘y’][1] − abs(room[‘dim’][‘y’][1] − room[‘dim’][‘y’][0]) / 2,
    room[‘dim’][‘z’][1] − abs(room[‘dim’][‘z’][1] − room[‘dim’][‘z’][0]) / 2,
  )
  cube.data.transform(Matrix.Translation(translation))
  mat = load_asset(room[‘material’][‘uuid’], repo_url)
  cube.data.materials.append(mat)
def create_light(item):
  light_data = bpy.data.lights.new(‘light’, type=item[‘type’])
  light = bpy.data.objects.new(‘light’, light_data)
  light.location = (item[‘location’][0], item[‘location’][1], item[‘location’][2])
  light.data.energy = item[‘energy’]
  bpy.context.collections.objects.link(light)
def setup_camera(camera):
  cam_data = bpy.data.cameras.new(camera[‘id’])
  cam = bpy.data.objects.new(camera[‘id’], cam_data)
  bpy.context.collection.objects.link(cam)
  cam.location = (camera[‘location’][0], camera[‘location’][1]), camera[‘location’][2]
  bpy.context.scene.camera = cam
def create_scene(scene_desc):
  create_room(scene_desc[‘room’])
  for light in scene_desc[‘lights']:
    create_light(light)
  for camera in scene_desc[‘cameras']:
    setup_camera(camera)
def clean_objects( ):
  for obj in bpy.data.objects:
    obj.delete( )
def place_object(item):
  if item[‘uuid’] not in local_asset_store:
    model = load_asset(item[‘uuid’], repo_url)
    if model is None:
      return None
    local_asset_store[item[‘uuid’]] = model
  else:
    model = local_asset_store[item[‘uuid’]]
  obj = bpy.data.objects.add(model)
  bpy.context.view_layer.objects.active = obj
  bpy.context.object.location = (item[‘location’][0], item[‘location’][1], item[‘location’][2])
  bpy.context.object.rotation_euler[2] = item[‘rotation’]
def render_image(camera_id, sample_count = 0):
  bpy.context.scene.render.filepath = os.path.join(output_dir, (render_file_pattern % (camera_id,
sample_count)))
  bpy.ops.render.render(animation=False, write_still=True, use_viewport=True, layer=‘’, scene=‘’)
def annotate_scene(camera_id, sample_count = 0):
  annotation_file = os.path.join(output_dir, (anno_file_pattern % (camera_id, sample_count)))
  create_annotation(bpy, camera_id, type=‘bounding_box’, file=annotation_file, format=‘CVAT for
Images 1.1’)
def main_loop( ):
  # This json file is generated by the scene description parser.
  f = open(‘sample_scene_description.json’)
  scene = json.load(f)
  create_scene(scene)
  for i in range(0, len(scene[‘objects'])):
    print(‘Render: ’ + str(i))
    clean_objects( )
    for item in scene[‘objects'][i]:
      if place_object(item) is None:
        raise Exception(‘cannot load model’)
    for camera in scene[‘cameras']:
      render_image(camera[‘id’], i)
      annotate_scene(camera[‘id’], i)
  print(‘-- Finished --’)
print(‘-- Start Main Loop --’)
main_loop(
```

Example Code of a Command File “sample_bInder_api_render.py”

The command file refers to a configuration file (as explained below), which contains all information relevant to the scene to be generated, and it can therefore be kept very generic and consequently be used for various scenes.

The exemplary code of the command file above is designed for execution on a processor using the “Blender” 3-D graphics suite and uses a corresponding Blender API (bpy) which itself can execute functions of the Blender program. In this case, the code refers to the configuration file, in this case “sample_scene_description.json”, and thus processes the defaults for the scene to be generated that are input by the user, as described below.

In a further step 11 of the method according to the disclosure, a user creates a preferably digital description of the entire content of a scene to be simulated in closed surroundings. For this description, it is possible to use both predefined elements and/or parameters and elements and/or parameters determined by the user. Predefined elements and/or parameters, such as descriptions of the surroundings, objects, persons, optionally with dedicated roles, materials and the like, can be stored in a corresponding database and thus be made available to the user. The predefined elements can be available as a description, as image data, and/or as combinations of a description and image data. Image data can be available as digital images of real elements, in rendered form, or as a combination of both. All parameters relevant to the scene to be simulated, objects, illumination, camera properties, object positions, properties of the surroundings, occlusion planes, and/or time-dependent changes of parameters and movements of objects are described in the description. The simulated movements lead to a dynamic scene, with the image data records preferably representing a video stream consisting of corresponding frames.

Accordingly, for example, the surroundings in which the scene should occur can be described, or an appropriate predefined description of the surroundings, for example the description of an operating theater, can be selected by the user. In this case, the description of the surroundings may comprise the dimensions, optionally windows and/or doors, light sources and the position of at least one camera, from the viewing angle of which the simulated scene should be represented, the camera resolution and image quality, and properties of the materials used in the surroundings, for example wall and floor materials. In this case, the description of the surroundings can be stored in the corresponding database as a predefined description for selection by the user, wherein this predefined description can be the description of a virtual room or a physically existing room. In the case of a physically existing room, the description may also comprise information obtained by an appropriate device for the three-dimensional capture of the room, for example by means of three-dimensional laser scanning or the like. In particular, it is also possible to capture digital images of a physically existing room, preferably in combination with appropriate depth information. In particular, such digital images can also be used as a part of or basis for the subsequent simulation of the scene. Moreover, it is also possible to extract camera properties, optical errors or disturbances of the optical system used to capture digital images of the physical surroundings, and use these as digital filters. In the simulation process, these digital filters can be applied to the rendered image material.

Predefined descriptions can be modified, augmented, created, and also stored for further use by the user.

For example, a specific operating theater in a hospital can be measured and can be stored in the form of a predefined description for further use. The predefined description of the operating theater may also comprise any desired further objects, for example operating theater furnishings, illumination, room cameras, fixedly installed equipment and the like, in addition to purely the dimensions. Moreover, as explained above, the predefined description may also contain digital images, especially with additional depth information, which can be used in part or as a basis for the subsequent simulation of the scene.

Following the selection of the surroundings for the simulation, the user can select further elements for the simulation from the database or describe these directly. Descriptions of individual elements for the simulation can likewise be modified, augmented, created, and also stored for further use by the user. For example, certain objects and equipment occurring in the scene to be simulated, and also persons, may be added. Using the example of the operating theater, it is possible to add persons such as, e.g., patients, cleaning staff, surgical technologists and/or physicians. In this case, the description may also take account of hospital-specific aspects such as the color of the apparel. Moreover, equipment required for the operation may be added and optionally described in greater detail. Moreover, it is also possible to describe the objects which should or may move in the simulation and the form this takes; the movements can either be defined specifically or carried out automatically and—preferably within certain specifications—randomly in the subsequent simulation.

Equally, more comprehensive scene descriptions can be stored as a predefined description for further use, and can be utilized to create new simulations. Thus—using the example of the operating theater once again—the instrumental and/or human resources for a specific procedure, for example a laparoscopy, can be selected and optionally adapted by the user.

Additionally, general parameters may be defined for the simulation. For example—if not already contained in the description—the type and location of the illumination and camera can be selected. If the simulation should be implemented on the basis of physical surroundings, for example a physical operating theater, the illumination and camera(s) can be matched to the situation in the physical surroundings. In this context, individual characteristics of the physical elements intended to be reproduced in the simulation can also be included. For example, the properties of the real camera can be taken into account. If a prediction model trained using the simulation data is intended to be subsequently applied in precisely these surroundings on the basis of images captured by the real camera, then the quality of the predictions can be increased by a preceding consideration of the camera properties.

The individual elements listed in the configuration file or introduced or selected by the user are preferably described with allowed and forbidden states. Accordingly, specific objects can be described as immobile, and others can be described as mobile and immobile. For example, a hospital bed can be defined as mobile but always standing on the floor, and modifiable in height within defined limits, whereas an operating table can be defined as stationary, standing on the floor, and modifiable in height. Certain groups of people can be assigned certain movement regions or occupancy regions depending on their roles. For example, using the example of the operating theater, a patient may lie on a hospital bed or on the operating table, whereas hospital staff cannot lie either on the bed or the table but can otherwise be arranged and moved freely in the operating theater in the simulation of the scene. The definition of allowed and forbidden states may have any desired level of detail; for example, certain pieces of equipment or persons may be assigned to specific possible positions for a certain medical procedure. For example, the positions of operating staff and equipment in the case of ENT operations differ significantly vis-à-vis the positions in the case of procedures on the feet. The definitions of the states may also have an inherent hierarchy; i.e., an object A may be arranged on an object B, but not vice versa. Accordingly, definitions of occlusion planes may also comprise hierarchic pieces of information. At this point, the user also has the option of defining individual elements in the description as constant; i.e., the only allowed state for this element is the state initially determined by the user in the description of the scene. Some predefined elements that are added to the description by the user, for example walls or securely installed furnishing or equipment, are defined as constant as a matter of principle. The definition of allowed and forbidden states allows the simulations to be designed to be more realistic since no scene that is precluded in reality is created. Equally, application-specific state definitions, for example the aforementioned distinction between an ENT operation and foot operation, may lead to specific prediction models with a correspondingly high prediction quality for the respective application.

Using a suitable computer program (parser), the inputs of the user are converted into a format more suitable for further processing, and hence the configuration file referred to in the command file is created, in a further step 12 of the method according to the disclosure. The formats possible in this context depend on the subsequently used computer program; a possible format used in the following exemplary code is JavaScript Object Notation (JSON), which is a compact data format with a simple readable text form for the data interchange between applications.

```
{
  “cameras”: [
    {
      “id”: “camera_1”,
      “location”: [−3.9, 3.1, 6.9]
    },
    {
      “id”: “camera_2”,
      “location”: [5.8, 3.1, 0.01]
    }
  ],
  “lights”: [
    {
      “id”: 1,
      “type”: “POINT”,
      “location”: [−4, 3.19, 3],
      “energy”: 200.0
    },
    {
      “id”: 2,
      “type”: “AREA”,
      “location”: [5.9, 3.19, 6.9],
      “energy”: 500.0
    }
  ],
  “room”: {
    “dim”: {
      “x”: [−4,6], “y”: [0, 3.2], “z”: [0,7]},
    “material”: {
      “id”: “6819f000-4575-41ec-809e-4ff5b366fb60”
    }
  },
  “objects”: [
    [
      {
        “id”: “20007117-7948-4e0b-8dba-25c0ca6d1c30”,
        “name”: “hospital_bed”,
        “class”: “transport-bed”,
        “location”: [3.4, 0.0, 2.5]
        “rotation”: 35
      },
      {
        “id”: “90dc80ce-782b-4d85-8562-891aca6b09f4”,
        “name”: “staff_1”,
        “class”: “person”,
        “location”: [−2.67, 0.0, 4.2],
        “rotation”: 237
      },
      }
        “id”: “3bdce5b5-07b8-484a-84c0-49e59d758283”,
        “name”: “staff_2”,
        “class”: “person”,
        “location”: [1.7, 0.0, −0.31],
        “rotation”: 56
      }
    ],
    [
      {
        “id”: “20007117-7948-4e0b-8dba-25c0ca6d1c30”,
        “name”: “hospital_bed”,
        “class”: “transport-bed”,
        “location”: [1.3, 0.0, 2.6],
        “rotation”: 43
      },
      {
        “id”: “90dc80ce-782b-4d85-8562-891 aca6b09f4”,
        “name”: “staff_1”,
        “class”: “person”,
        “location”: [−2.1, 0.0, 1.67],
        “rotation”: 211
      }
    ]
  ]
}
```

Example Code of a “sample_scene_description.json” Configuration File

The exemplary code of a configuration file given above describes a simple scene in a hospital room with two people and a hospital bed.

Optionally, a database for objects or materials, in particular for objects and/or materials described for three-dimensional representations, can be created in a further step 13 of the method according to the disclosure. In this database, it is possible to store descriptions and/or representations of standardized objects and/or materials, which can be used when creating the image data by appropriate pointers or references in the configuration file.

Referring back to FIG. 1, the command file can in a further step 14 of the method according to the disclosure be transferred to the processor for execution and for the creation of an image data record of the scene to be simulated. The execution of the command file can be implemented by or together with the execution of a computer program for creating CGI, for example Blender. The transfer of the command file to the processor can be implemented together with the configuration file, or the latter can alternatively be read when the command file is executed. Provided the configuration file contains references to predefined, specified objects or materials, in particular objects and/or materials described for three-dimensional representations, the corresponding data associated with these objects and/or materials can in a further step 16 of the method according to the disclosure be retrieved from the corresponding database. As explained above, these data may comprise description, image data and/or combinations of description and image data.

Then, in a further step 17 of the method according to the disclosure, the scene described by the configuration file can be generated on the processor, optionally under the execution of a computer program for creating CGI, and an image data record can be created in step 18. This image data record is a representation of a scene in accordance with the configuration file. The individual images of the image data record are two-dimensional representations of the scene at a respective time and from the viewing angle of the camera described in the configuration file. In the temporal progression of scenes, frames which when arranged in temporal sequence represent an appropriate video stream are created at appropriate times. Provided the configuration file describes a plurality of cameras, it is possible to create different image data records which correspond to the respective viewing angles of the cameras. In this case, the individual image data records can be correlated by way of appropriate time assignments; i.e., processes in a scene can be captured from different camera perspectives. If the simulation is based on an augmented command file with associated objects and/or materials stored with corresponding image data, then these image data are integrated in the simulation of the scene and used accordingly for the creation of the image data records. For example, if a scene is in a physically existing operating theater, which is stored with appropriate image material, then the scene can be augmented with the image material. This allows hybrid image data records, i.e., image data records consisting of real and rendered data, to be created, whereby the heterogeneity of the image material can be increased in comparison with entirely rendered image data records, and hence the value as training data can be increased. Moreover, it is also possible to apply filters to provide the rendered image material with a more realistic appearance. In particular, the filters created on the basis of camera characteristics identified in real images can be applied to rendered image material in order thus to create a more realistic or more natural appearance.

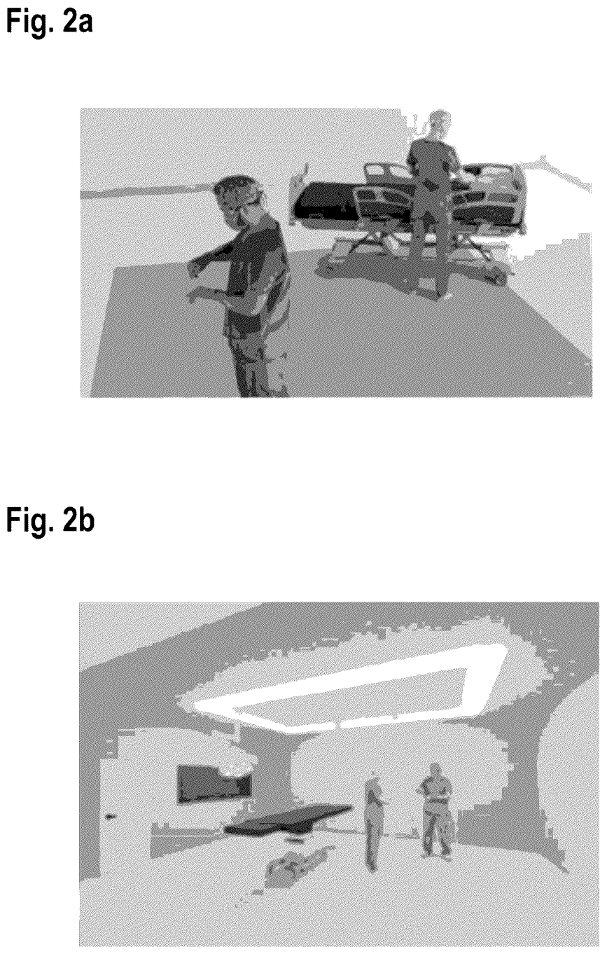

FIGS. 2a and 2b show images of two scenes in different operating theaters by way of example. In this case, FIG. 2a shows two members of the hospital staff and an empty hospital bed in an operating theater, with the hospital bed being partly obscured by one of the individuals. Here, the floor of the operating theater features a two-color design.

FIG. 2b shows an operating theater with two members of the hospital staff, an empty operating table, two surgical lamps, rectangular ceiling lighting, a door, and a rectangular, non-illuminated, and hence black display unit on the wall.

Further with reference to FIG. 1, annotation files associated with the image data records can also be created in addition to the creation of the image data records in a further step 19 of the method according to the disclosure. Like in the exemplary code of the command file above, this procedure can likewise be defined for application in the command file (in this case “create_annotation (bpy, camera_id, type=‘bounding_box’, file=annotation_file, format=‘CVAT for Images 1.1’)”); in this example, the spatial coordinates of the scene are stored in the ‘bpy.context’ instance. These coordinates, together with the camera, enable the calculation of the 2-D image coordinates for the annotation file. Accordingly, the annotation file can be created together with the creation of the image data or, alternatively, at a subsequent point in time using the image data. The annotation files can be created in various formats, like for example the “CVAT for Images” used in the following exemplary code.

```
<?xml version=“1.0” encoding=“utf-8”?>
<annotations>
  <version>1.1</version>
  <meta>
    <task>
      <id>76</id>
      <name>Blender Sample Scene</name>
      <size>1</size>
      <mode>annotation</mode>
      <overlap>0</overlap>
      <bugtracker></bugtracker>
      <created>2023-02-14 13:28:21.787704+00:00</created>
      <updated>2023-02-14 13:29:09.809934+00:00</updated>
      <subset>default</subset>
      <start_frame>0</start_frame>
      <stop_frame>0</stop_frame>
      <frame_filter></frame_filter>
      <segments>
        <segment>
          <id>394</id>
          <start>0</start>
          <stop>0</stop>
          <url>http://10.3.4.9/?id=394</url>
        </segment>
      </segments>
      <owner>
        <username>labeler</username>
        <email></email>
      </owner>
      <assignee></assignee>
      <labels>
        <label>
          <name>transport-bed</name>
          <color>#cc3366</color>
          <attributes>
          </attributes>
        </label>
        <label>
          <name>person</name>
          <color>#24b353</color>
          <attributes>
          </attributes>
        </label>
      </labels>
    </task>
    <dumped>2023-02-14 13:29:21.934285+00:00</dumped>
  </meta>
  <image id=“0” name=“or_sample_camera_1_1.jpg” width=“1280” height=“720”>
    <box label=“person” occluded=“0” source=“manual” xtl=“653.29” ytl=“114.57” xbr=“719.27”
ybr=“292.83” z_order=“0”>
    </box>
    <box label=“person” occluded=“0” source=“manual” xtl=“446.09” ytl=“140.03” xbr=“531.75”
ybr=“406.27” z_order=“0”>
    </box>
    <box label=“transport-bed” occluded=“0” source=“manual” xtl=“267.83” ytl=“328.71”
xbr=“770.21” ybr=“718.81” z_order=“0”>
    </box>
  </image>
</annotations>
```

Example Code of a “sample_annotations_cvat.xml” Annotation File

These annotation files are essentially based on the descriptions in the respective configuration files. Commenting comprises the categorization and labeling of the image data records so that these can be processed by a machine as training data, and so the image data records are thus rendered machine acquirable. The commenting adds information to each image accordingly, with regards to what can be seen where in the respective image. Since comprehensive information, for example position, dimension, etc., is available for each object, it is possible to create very precise comments. In contrast to the commenting of conventional image data, information about the entire content of the scene, i.e., about the surroundings, parameters, objects, etc., is available according to the present disclosure. Accordingly, the commenting can automatically be implemented with virtually any desired level of granularity. The categorization can also be implemented automatically on the basis of the configuration files since the content of the scenes is known. On the basis of the command file or the augmented command file, an appropriate annotation file can be created for each frame of an image data record.

If variations of the configuration files were created, then it is also possible to create annotation files for the command files and image data records resulting therefrom. The image data records can be created in various formats known to a person skilled in the art, for example Yolo, COCO or Pascal-VOC. Together with the associated annotation files, the image data records represent structured training data records.

Still referring to FIG. 1, certain parameters of the configuration file, preferably appropriately predefined parameters of the configuration file, can be varied in a further step 20 of the method according to the disclosure. Accordingly, it is for example possible to modify the arrangement and/or movement of certain objects, and/or the illumination can be modified. The method according to the disclosure is configured such that random changes can be made in the configuration file, and further image data records are created on the basis of these modified configuration files. Accordingly, image data records of similar scenes with the same content can be created by varying the configuration files. The random modifications of the configuration files are preferably implemented within the defined, allowed and forbidden states. For example, movements and/or locations of persons can be varied within the defined states. Equally, it is also possible to vary the movement of objects, for example a hospital bed and/or surgical lamps in an operating theater, within the defined states. As a result of these variations, it is possible to create image data records with different obscurations, illumination and shadowing effects, and positions of objects, as may also occur in a real scene with the same content and in the same surroundings. In principle, the user can determine the elements intended for variation. However, the variation is preferably implemented within the scope of the predefined states. As a result of this step of the method, the diversity of the image data records and hence the heterogeneity of the training data can be increased automatically—on the basis of an initial description of a scene by the user—and also the human bias possibly emerging from the description by the user can be reduced. Overall, this can consequently be conducive to the suitability of the created image data records as training data.

The training data records created using the method according to the disclosure can train prediction algorithms, for example object recognition algorithms for the application in similar surroundings or in the real surroundings defined in the configuration files. Once surroundings-specific prediction algorithms have been created, they can be selected for the specific application. For example, an object recognition algorithm created for a specific operating theater can be selected for the purpose of monitoring the corresponding operating theater. In an alternative to that or in addition, an image of the room obtained by a room camera arranged in the operating theater can be evaluated, and hence the respective operating theater can be recognized and the associated algorithm trained for this theater can be selected and used for room monitoring. Equally, specific algorithms can also be ascertained, either manually or automatically, on the basis of other parameters, for example the type of medical procedure, either by way of objects and/or persons present or in conjunction with further information, for example an operating schedule. As a result of the specifically trained prediction algorithms, it is possible to improve the predictions in the respectively associated surroundings.

Object detection models can be trained on the basis of image data records and annotation files created according to the disclosure.

In an application of the teaching according to the disclosure, created image data records were created together with annotation files for a simple operating theater scene with 2 members of staff and a patient trolley. The members of staff and the patient trolley were moved automatically within the room. 500 images were rendered automatically in accordance with a corresponding command file for two room cameras with different fields of view, wherein images in which the patient trolley was covered in full or in part by the members of staff were also generated. An example of one of these images is shown in FIG. 2Xa. Since all objects and movements were known as a result of the command file or configuration file, it was possible to create detailed annotation files. Using the created data, it was possible to significantly increase the detection accuracy of an object detection model (YoloX) that had been trained previously with a limited training data record based on real image material.

The scope of this disclosure includes all changes, replacements, variations, developments and modifications of the exemplary embodiments described or explained herein which would be understood by a person of average skill in the art. The scope of protection of this disclosure is not limited to the exemplary embodiments described or explained herein. Even though this disclosure comprehensively describes and explains the respective embodiments herein as specific components, elements, features, functions, operations or steps, any of these embodiments can moreover comprise any combinations or permutations of any components, elements, features, functions, operations or steps described or explained anywhere herein, which would be understood by a person of average skill in the art. A reference in the appended claims to the fact that a method or a device or a component of a device or a system is adapted, set up, capable, configured, able, operative or operational for the purpose of performing a specific function also includes this device, this system or this component, independently of whether it or this specific function is activated, switched on or enabled for as long as this device, this system or this component is adapted, set up, capable, configured, able, operative or operational to this end. Even though this disclosure describes or explains that certain embodiments provide certain advantages, certain embodiments may moreover provide none, some or all of these advantages.

1. A computer-implemented method for synthesizing application-specific image data for training artificial intelligence-based object recognition in images of medical and/or clinical workflows, comprising:
receiving a command file for execution on a processor to create image data records comprising up to three dimensions using at least one configuration file;
creating a configuration file describing a scene to be simulated, the configuration file defining one or more object parameters;
executing the command file and the configuration file on the processor to create a first image data record, the first image data record including a first object defined by the one or more object parameters;
creating a first annotation file associated with the first image data record;
storing the first image data record together with the first annotation file;
randomly modifying the configuration file by varying the one or more object parameters;
executing the modified configuration file on the processor to generate a second image data record, the second image data record including a second object different from the first object.

2.-15. (canceled)

16. The method as set forth in claim 1, wherein executing the command file and the configuration file on the processor to create the first image data record comprises receiving the first object from an object database based on the one or more object parameters.

17. The method as set forth in claim 1, wherein the first image data record and the second image data record each include a hospital room.

18. The method as set forth in claim 17, wherein the first object is a camera, the one or more parameters defining a perspective of the camera.

19. The method as set forth in claim 18, wherein the second object is the camera, the one or more parameters defining a different perspective of the camera than the first object.

20. The method as set forth in claim 18, wherein the first image data record includes a two-dimensional individual image, the individual image taken from a viewing angle of the camera.

21. The method as set forth in claim 1, wherein the one or more parameters comprise an inherent hierarchy of objects stored in an object database.

22. The method as set forth in claim 1, wherein the one or more parameters comprise states of defined, allowed, and forbidden.

23. The method as set forth in claim 1, wherein the configuration file describes a static or dynamic scene.

24. The method as set forth in claim 1, wherein the configuration file comprises general parameters, objects, materials, illumination, camera properties, object positions, object movements, properties of surroundings, occlusion planes, or time-dependent parameter changes.

25. The method as set forth in claim 1, wherein the scene described in the configuration file should be simulated in predetermined surroundings, the predetermined surroundings including a description of an operating room.

26. The method as set forth in claim 25, wherein the description of the operating room is captured by way of a scanning device or comprises digital images of the room.

27. The method as set forth in claim 24, wherein the general parameters comprise a description of the scene to be simulated.

28. The method as set forth in claim 1, wherein further image data records and annotation files associated with the further image data records are created on the basis of variations defined by the random modifications of the configuration file.

29. The method as set forth in claim 28, wherein the configuration file contains allowed and forbidden states of objects and illumination, and the variations are defined by these states.

30. The method as set forth in claim 28, wherein the variations relate to at least one light source.

31. The method as set forth in claim 1, wherein the image data records created and the annotation files associated therewith are stored in categorized fashion.

32. The method as set forth in claim 1, wherein the configuration file, at least in part, contains pieces of information based on a default.

33. A method for creating an application-specific, artificial intelligence-based object recognition, comprising the training of a prediction algorithm with training data synthesized according to claim 1.

34. A method for recognizing objects in a specific application, wherein a prediction algorithm trained with training data synthesized according to claim 1 is applied to at least one digital image and wherein the at least one image is an image of a scene which forms the basis of the training data created.
